(19) United States
(12) Patent Application Publication
(10) Pub. No.: US 2012/0271179 A1
(43) Pub. Date: Oct. 25, 2012

(54) FEMORAL COMPRESSION DEVICE

(75) Inventor: Tobias Adenmark, Uppsala (SE)

(73) Assignee: St. Jude Medical System AB, Uppsala (SE)

(21) Appl. No.: 13/517,163

(22) PCT Filed: Dec. 15, 2010
(86) PCT No.: PCT/EP10/69821

§ 371 Date: Jun. 19, 2012

Related U.S. Application Data
(60) Provisional application No. 61/288,988, filed on Dec. 22, 2009.

(30) Foreign Application Priority Data
Dec. 22, 2009 (SE) 0951002-5

(51) Int. Cl. A61B 5/022; A61B 5/0235

(52) U.S. Cl. 600/498; 600/499

(57) ABSTRACT

A femoral compression device (21) for compressive bearing 27b against the femoral artery of a patient, comprising a base plate (22); an inflatable air cushion (24) attached to the base plate; a first extension (23a) extending from a first side of the base plate; a second extension (23b) extending from a second side of the base plate, said second side being opposite to the first side; a belt (25) adapted to be attached to an end of the first extension and being adapted to extend around the patient's body to be attacked to an end of the second extension; a pump (26) for inflation of the inflatable air cushion; an air connection (27a, 27b, 27′) with a first end and a second end and connecting the pump at a first end and the air cushion at the second end, and a manometer (28) arranged for measurement of the current pressure difference between pressure prevailing inside the air connection and ambient air pressure. The manometer is arranged at the air connection separately from the pump and located such that a shut-off device (31) designed to close off the air connection may be arranged between said manometer and said pump.

### FIELD OF THE INVENTION

The invention relates generally to a femoral compression device, and in particular to a femoral compression device enabling arrangement of a shut-off device for closing an air connection connecting a pump with an inflatable pressuring means, and further comprising a pressure gauge.

### BACKGROUND OF THE INVENTION

Femoral compression devices for applying pressure on a patient's femoral artery after completion of an interventional procedure are known. An example of such a femoral compression device is disclosed in the U.S. Pat. No. 5,307,811, which is assigned to the present assignee.

A femoral compression device according to this patent publication comprises basically a pressurizing means for compressive bearing against a puncture site at a femoral artery of a patient, a belt adapted to be fixed around the patient's body, and a base plate supporting the pressurizing means and being provided with two extensions. In use, the pressurizing means, which in one embodiment has the form of an inflatable semi-spherical air cushion, is positioned over the femoral artery, and the belt, which extends from the end of the first extension, around the patient's body and to the end of the second extension, is tightened. To apply pressure on the femoral artery, the inflatable semi-spherical air cushion is inflated by a pump to a certain predetermined pressure, which is readable from a pressure gauge.

In the above mentioned patent, the pressure gauge comprises a mechanical manometer with an analog pointer. In the published U.S. Patent Application No. 2008/0319328, which is assigned to the present assignee, a pressure gauge in the form of an electronic manometer having a digital display is disclosed.

A common feature for both types of devices is that the electronic or mechanical manometer is placed in immediate vicinity of the pump; that is, the manometer and the pump can in fact be regarded as an integrated unit.

Although not actually called for, many users, e.g. nurses or other medical personnel, prefer to close off the air connection by means of a separate shut-off device, typically a clamp; and, as a response to this practice, a femoral compression device, which by the present applicant is sold under the registered trademark FemoStop®, is in one version delivered with a plastic clamp, which—if it is used—is placed between the inflatable air cushion and the integrated pump and manometer, and is clamped together, to thereby close off the air connection between the pump and the inflatable air cushion. Although this procedure is regarded as a security measure, to ensure that air pressure within the air cushion is maintained even in the very unlikely event that the pump should be leaking, this procedure has a drawback. When the manometer no longer is operatively connected to the inflatable air cushion, the displayed pressure reading has no longer any significance, i.e. the pressure reading will not show the actual pressure prevailing within the inflatable air cushion. The displayed pressure will, however, not necessarily be zero, but will show the air pressure prevailing in the air connection and/or in the pump, which, to the inexperienced user, can be misleading. If, for example, air should leak out from the inflatable air cushion, this pressure drop would not be displayed on the manometer, as the air connection between the air cushion and the manometer is closed off by the clamp.

Consequently, there is still a need for an improved femoral compression device comprising a pressure gauge, which obviates the drawbacks associated with femoral compression devices according to the prior art, and the object of the present invention is to remove those drawbacks.

### SUMMARY OF THE INVENTION

The above-mentioned object is achieved by the present invention according to the independent claims.

Preferred embodiments are set forth in the dependent claims.

Embodiments of the invention are directed to a femoral compression device with a pump, an air connection connecting the pump to an inflatable air cushion, a pressure gauge, and a shut-off device. In contrast to the femoral compression devices hitherto known, the pressure gauge is not integrated with the pump but is a separate pressure gauge, which, in the air connection, is positioned between the air cushion and the shut-off device, or in connection to the air cushion. By means of this design, the pressure gauge will always measure the true pressure prevailing within the air cushion, regardless whether the shut-off device is closed or open.

In embodiments of the present inventions, the pressure gauge can be in the form of a mechanical manometer or an electronic manometer; in each case provided with a digital or analog display. The air connection between the pump and the inflatable air cushion is typically a tube made from rubber, plastic or metal, while the shut-off device typically is a clamp, which is arranged on the outside of the air connection tube, but can also be in the form of a stopcock or stop valve, which, at least partly, is arranged within the air connection tube.

Herein, two general embodiments comprising a tube, an electronic and digital manometer, and a clamp will be described, but the alternatives indicated above are within the scope of the present invention.

### BRIEF DESCRIPTION OF THE DRAWINGS

FIG. 1 illustrates schematically a femoral compression device according to the prior art.

FIG. 2 illustrates schematically a femoral compression device with an electronic manometer arranged between an inflatable air cushion and a pump in accordance with one embodiment of the present invention.

FIG. 3 illustrates schematically a femoral compression device with an electronic manometer arranged between an inflatable air cushion and a pump in accordance with a second embodiment of the present invention.

### DETAILED DESCRIPTION OF PREFERRED EMBODIMENTS

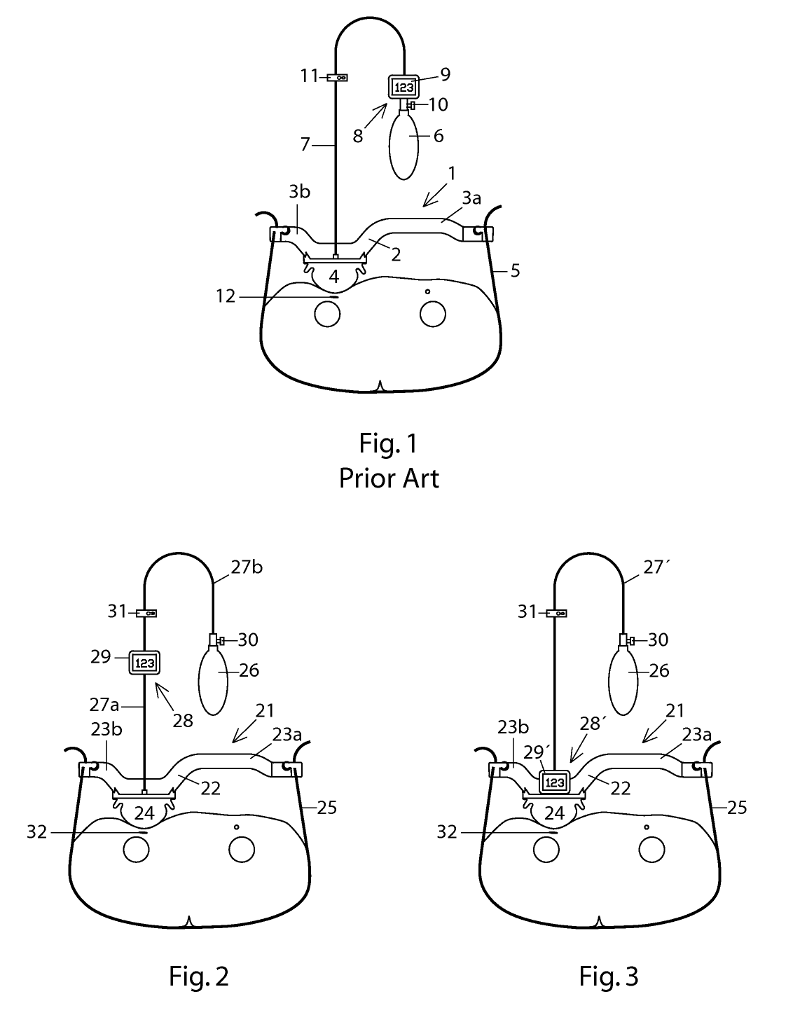

In FIG. 1, a femoral compression device 1 according to the prior art is schematically illustrated. The femoral compression device 1 comprises a base plate 2 with two extensions 3a and 3b, an inflatable air cushion 4, a belt 5, a pump 6, an air connection 7, and an electronic pressure gauge or manometer 8 with a display 9. Except for the manometer 8 and possibly the pump 6, the femoral compression device 1 is of the same construction as was disclosed in the previously mentioned patent U.S. Pat. No. 5,307,811. In use, the inflatable and semi-spherical air cushion 4 is positioned over the femoral artery 12 of a patient, and the belt 5, which extends from the end of the first extension 3a, around the patient's body and to the end of the second extension 3b, is tightened and secured by belt fasteners at the end of each extension. To apply pressure on the femoral artery, the inflatable semi-spherical air cushion 4 is inflated by the pump 6 to a certain pressure, which is measured by the manometer 8 and displayed on the display 9. The pump 6 comprises further a vent knob 10 used for pressure regulation and venting of the system. Optionally, the femoral compression device 1 may further be provided with a shut-off device 11, typically a clamp 11, which then is clamped around air connection 7, to close off the connection between the inflatable air cushion 4 and the pump 6 with manometer 8. When the clamp 11 is closed, the manometer 8 is no longer operatively connected to the inflatable air cushion 4, and the display 9 will not show the actual pressure prevailing within air cushion 4.

FIG. 2 shows schematically a femoral compression device 21 according to one embodiment of the present invention. The femoral compression device 21 comprises a base plate 22 with two extensions 23a and 23b, an inflatable air cushion 24, a belt 25, a pump 26, an air connection, which consists of a first portion 27a and a second portion 27b, and an electronic pressure gauge or manometer 28 with a display 29. The pump 26 comprises further a vent knob 30 used for pressure regulation and venting of the system. The femoral compression device 21 is further provided with a shut-off device 31, in this case a clamp 31, which is clamped around the air connection, to thereby effectively divide the air connection into two portions, i.e. the first portion 27a and the second portion 27b. In use, the femoral compression device 21 functions like the femoral compression device 1, which was described above in conjunction with FIG. 1. There is, however, one notable difference between compression device 1 and compression device 21. The manometer 28 of the femoral compression device 21 is not integrated with the pump 26. Instead, the manometer 28 is a separate element, and is positioned in the air connection such that the shut-off device 31 is located between the pump 26 and the manometer 28, or—alternatively expressed—such that the manometer 28 with the display 29 is positioned between the inflatable air cushion 24 and the shut-off device 31.

In one embodiment the manometer is located at essentially the same distance from the first and second ends of the air connection.

In another embodiment the manometer is located less than 20 cm from the first end, i.e. from the pump 26.

FIG. 3 discloses still another embodiment of the present invention where the manometer 28′ with display 29′ is arranged in connection to the inflatable air cushion 24. In this embodiment the air connection 27′ runs all the way to the air cushion. The manometer may e.g. be arranged in an indentation of the base plate 22. For a detailed description of the remaining parts of this embodiment it is referred to the description of the embodiment illustrated in FIG. 2. This embodiment is particularly advantageous in that it is impossible to arrange a shut-off device between the air cushion and the manometer.

By having a manometer 28, 28′, which is separated from the pump 26 and is positioned between the shut-off device 31 and the inflatable air cushion 24, the display 29, 29′ will always display the actual air pressure prevailing within air cushion 24, regardless whether or not shut-off device 31 is closed or open. By this separation of pump 26 and manometer 28, 28′ and the positioning of the clamp 31 between the manometer 28, 28′ and the pump 26, a safer and more user-friendly femoral compression device has been provided.

As has been indicated above, a shut-off device can in accordance with the present invention be any kind of device which is capable of shutting off the air connection between the pump and the inflatable air cushion. A clamp is only an exemplifying device, but other devices, such a valves or stopcocks, are possible. A shut-off device can be arranged on the outside of the air connection or can be arranged within the air connection, and may be fixedly arranged to the air connection or may be a separate device. In one embodiment the shut-off device is a valve fixedly arranged at the air connection and provided with a closure portion used to open and close the valve.

The present invention also relates to an inflatable air cushion unit adapted for attachment to a femoral compression device as described above. The inflatable air cushion unit comprises an inflatable air cushion 24, a pump 26 for inflation of the inflatable air cushion, an air connection 27a, 27b, 27′ with a first end and a second end and connecting the pump at a first end and the air cushion at the second end. The unit further comprises a manometer 28, 28′ arranged for measurement of the current pressure difference between pressure prevailing inside the air connection and ambient air pressure. The manometer is arranged at the air connection separately from the pump and located such that a shut-off device 31 designed to close off the air connection may be arranged between said manometer and said pump. All features described above in relation to the two embodiments of the femoral compression device are equally applicable in connection with the air cushion unit.

Although the present invention has been described with reference to specific embodiments, also shown in the appended drawings, it will be apparent for those skilled in the art that many variations and modifications can be done within the scope of the invention as described in the specification and defined with reference to the claims below.

1.-11. (canceled)

12. A femoral compression device for compressive bearing against the femoral artery of a patient, comprising:
a base plate;
an inflatable air cushion attached to the base plate;
a first extension extending from a first side of the base plate;
a second extension extending from a second side of the base plate, the second side being opposite to the first side;
a belt adapted to be attached to an end of the first extension and being adapted to extend around the patient's body to be attached to an end of the second extension;
a pump for inflation of the inflatable air cushion;
an air connection with a first end and a second end and connecting the pump at the first end and the air cushion at the second end, and
a manometer arranged for measurement of the current pressure difference between pressure prevailing inside the air connection and ambient air pressure, the manometer arranged at the air connection separately from the pump and located such that a shut-off device designed to close off the air connection may be arranged between the manometer and the pump.

13. A femoral compression device according to claim 12, further comprising the shut-off device.

14. A femoral compression device according to claim 13, wherein the shut-off device is designed to close off the air connection at a first point along the air connection; and the manometer is located at a second point along the air connection, such that the second point is closer to the second end than the first point.

15. A femoral compression device according to claim 12, wherein the manometer is an electronic manometer.

16. A femoral compression device according to claim 12, wherein the manometer is provided with a display.

17. A femoral compression device according to claim 13, wherein the shut-off device is a clamp.

18. A femoral compression device according to claim 13, wherein the shut-off device is a valve fixedly arranged at the air connection and provided with a closure portion used to open and close the valve.

19. A femoral compression device according to claim 12, wherein the manometer is located at essentially the same distance from the first end and from the second end of the air connection.

20. A femoral compression device according to claim 12, wherein the manometer is located less than 20 cm from the first end.

21. A femoral compression device according to claim 12, wherein the manometer is mounted on the inflatable air cushion.

22. An inflatable air cushion unit adapted for attachment to a femoral compression device, the inflatable air cushion unit comprising:
an inflatable air cushion;
a pump for inflation of the inflatable air cushion;
an air connection with a first end and a second end and connecting the pump at the first end and the air cushion at the second end;
a shut-off device configured to close off the air connection; and
a manometer arranged for measurement of the current pressure difference between pressure prevailing inside the air connection and ambient air pressure, the manometer arranged at the air connection separately from the pump and located such that the shut-off device is arranged between the manometer and the pump.

23. An inflatable air cushion unit according to claim 22, wherein the manometer is mounted on the inflatable air cushion.

24. A method for applying compression against a femoral artery, comprising the steps of:
arranging an inflatable air cushion, a pump for inflation of the air cushion, an air connection between the pump and the air cushion, and a manometer operatively between the pump and the air cushion such that the air cushion is configured to apply compression to a femoral artery;
inflating the air cushion using the pump to apply compression to the femoral artery; and
isolating the pump from the manometer using a shut-off device configured to close off a portion of the air connection.

25. A method according to claim 24, wherein the inflatable air cushion is connected to first and second extensions via a base plate, each of the first and second extension having a device for affixing a belt at an end, and wherein the method further comprises the step of adjusting the belt to extend around a patient's body.

26. A method according to claim 24, wherein the manometer is mounted on the inflatable air cushion.

27. A femoral compression device according to claim 12, wherein the manometer is in fluid communication with the air cushion.
